(19) United States
(12) Patent Application Publication
(10) Pub. No.: US 2007/0193882 A1
(43) Pub. Date: Aug. 23, 2007

(54) ELECTROCHEMICAL TEST STRIP FOR MULTI-FUNCTIONAL BIOSENSOR

(76) Inventors: Ken-Shwo Dai, Taipei (TW); Chia Chi Lin, Taipei (TW)

Correspondence Address: BAKER & MCKENZIE LLP, Pennzoil Place, South Tower, 711 Louisiana, Suite 3400, HOUSTON, TX, 77002-2716, US

(21) Appl. No.: 11/426,076

(22) Filed: Jun. 23, 2006

(30) Foreign Application Priority Data
Feb. 21, 2006 (TW) 095202876

(51) Int. Cl. G01N 33/487

(52) U.S. Cl. 204/403.02

(57) ABSTRACT

The present invention provides an electrochemical biosensor test strip used for quantitative determination of an analyte in a liquid sample. Methods of fabricating the test strip and reagent formulas are also provided. Improvements in electrochemical test strips are required to detect the presence of a compound in a liquid mixture using a smaller sample size with increased accuracy. The present invention was developed to be user-friendly, decrease sample requirements and decrease analyzing time while increasing the reproducibility and accuracy of the electrochemical test strip.

### CROSS-REFERENCE TO RELATED APPLICATIONS

This application claims priority to Taiwan Patent Application 095202876, filed Feb. 21, 2006, the disclosure of which is incorporated by reference in its entirety herein.

### FIELD OF THE INVENTION

The invention relates to a test strip. More particularly, the invention relates to a disposable, electrochemical, biosensor test strips; preparation process thereof, and uses of the same for quantification of analyte in a liquid sample.

### BACKGROUND OF THE INVENTION

Diabetes mellitus (DM) and its related complications have been known to be the major health problems worldwide. A statistical analysis showed that diabetic population increases yearly and an estimation of 370 million people with diabetes was expected by 2030 (World Health Organization 2003). It has been reported that the maintenance of near-normal blood glucose level is important in reducing the risk or slowing the progress of diabetes-related complications/deaths (World Health Organization 2003; The Diabetes Control and Complications Trial Research Group (1993) N Engl J Med 329: 977-986). Thus, self-monitoring blood glucose device was recommended as one of the important tools for diabetic patients to control their blood glucose levels (Sonksen et al. (1978) Lancet 1: 729-732; Walford et al. (1978) Lancet 1: 732-735; Ikeda and Tsuruoka (1994) Diabetes Res Clin Pract Suppl: S269-271; Evans (1999) BMJ 319: 83-86; Franciosi (2001) Diabetes Care 24: 1870-1877). Over the past few years, many handheld glucose meters that employed disposable electrochemical test strips have been developed and introduced into the self-monitoring diagnostic market. The clinical accuracy of many of these glucose measurement systems has been evaluated (Tate et al. (1992) Diabetes Care 15: 536-538; Kilpatrick et al. (1994) Diabet Med 11: 214-217; Trajanoski et al. (1996) Diabetes Care 19: 1412-1415; Arens et al. (1998) Clin Chem Lab Med 36: 47-52; Rheney and Kirk (2000) Ann Pharmacother 34: 317-321; Solnica et al. (2001) Clin Chem Lab Med 39:1283-1286; Alto et al. (2002) J Am Board Fam Pract 15:1-6; Demers et al. (2003) Am J Health Syst Pharm 60:1130-1135; Dai et al. (2004) Clin Chim Acta. 349: 135-41).

Electrochemical test strips (strips containing an electrode system) have been extensively applied in self-monitoring systems for the measurement of analyte levels in liquid samples. U.S. Pat. No. 5,120,420 (Nankai et al. 1992) discloses an electrochemical test strip comprises an insulating base board, an electrode system, an insulating layer and a reaction layer. By integrating an insulating base board with a spacer and a cover, a space including a reaction layer is formed. U.S. Pat. No. 5,288,636 (Pollmann et al., 1994) disclosed an electrochemical test strip containing a pair of electrodes (working and counter) of the same size. The electrodes are made of the same electrically conducting materials and supported on the first electrical insulator. U.S. Pat. No. 5,437,999 (Diebold et al. 1995) discloses the fabrication of high-resolution electrodes based on the novel adaptation of some techniques common to the PCB industry. U.S. Pat. No. 5,762,770 (Pritchard et al. 1998) discloses an electrochemical biosensor test strip that has a minimum volume blood sample requirement of about 9 microliters. U.S. Pat. No. 5,727,548 (Hill et al. 1998) discloses an enzymatic sensing electrode formed by screen printing the enzyme onto a substrate. The two-electrode strip comprises an elongated support adapted for releasable attachment to the readout circuitry.

Various electrochemical test strips have been developed and used to detect the presence of a compound in a liquid mixture. Future consideration on developing a user-friendly, little sample requirement, reproducible, short analyzing time and accurate electrochemical test strip is necessary

### SUMMARY OF THE INVENTION

According to the present invention, an electrochemical biosensor test strip used for quantitative determination of an analyte in a liquid sample is disclosed. The electrochemical biosensor test strip comprises: 1) a first insulating base plate; 2) at least two conductive tracks in a lengthwise direction printed on the first insulating base plate; 3) a second insulating base plate overlaid on the first insulating base plate; 4) a reaction area defined by the U-shaped cutout of the second insulating base plate; 5) a reagent film deposited in the reaction area for reacting with an analyte in a liquid sample; 6) a cover disposed atop the second insulating base plate.

According to one aspect of the present invention, a method for fabricating a biosensor test strip is provided comprising the steps of: 1) printing the first insulating base plate with conductive tracks (a pair of electrodes and a plurality of electrical contacts); 2) overlaying a second insulating base plate on the first insulating base plate; 3) coating a mixture of reagent over the reaction area to form a reagent film; and 4) depositing a cover onto the second insulating base plate.

According to another aspect of the present invention, the formula of a mixture of reagent for making a reagent film is provided comprising at least one biological active substance (e.g., enzyme), and other components such as mediator, surfactant, and at least one binder.

According to the third aspect of the present invention, a reaction chamber is formed between the cover and the reagent film. The reaction chamber requires a lower volume of liquid sample which is about 2.5 microliters. The liquid sample is introduced into the reaction chamber from a small semicircle-shaped cutout on the top end of the cover. The cover includes an open slot, an air vent opening over the bottom end of the reaction chamber, to reduce the air bubbles trapped in the reaction chamber. The action of capillary force for introducing the liquid sample into the reaction chamber is defined by the thickness of the second insulating base plate.

According to the fourth aspect of the present invention, the biosensor test strip employs at least two conductive tracks (a pair of electrodes with a plurality of electrical contacts) formed on the first insulating base plate. The electrodes include a working electrode and a reference electrode which are separated apart from each other. One of the electrical contacts is electrically connected to the conductive track associated with the working electrode. The rest of electrical contacts are electrically connected to the conductive track associated with the reference electrode comprising at least one turn-on/type recognition electrical contact.

### BRIEF DESCRIPTION OF THE DRAWINGS

FIG. 1 is an exploded perspective view of a test strip according to the present invention.

FIG. 2 is a top perspective view of the test strip of FIG. 1;

FIG. 3 is a front view of the test strip of FIG. 1;

FIG. 4 is a correlation curve of the concentration readings using electrochemical biosensor test strips of the present invention versus the concentration readings of the same samples determined using a YSI glucose analyzer.

### DETAILED DESCRIPTION OF THE INVENTION

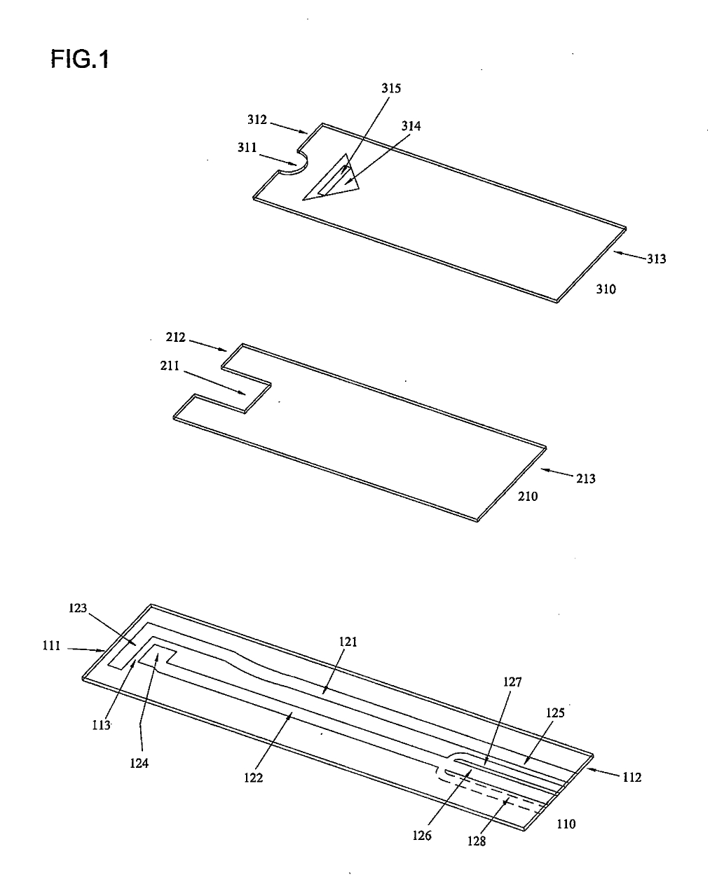

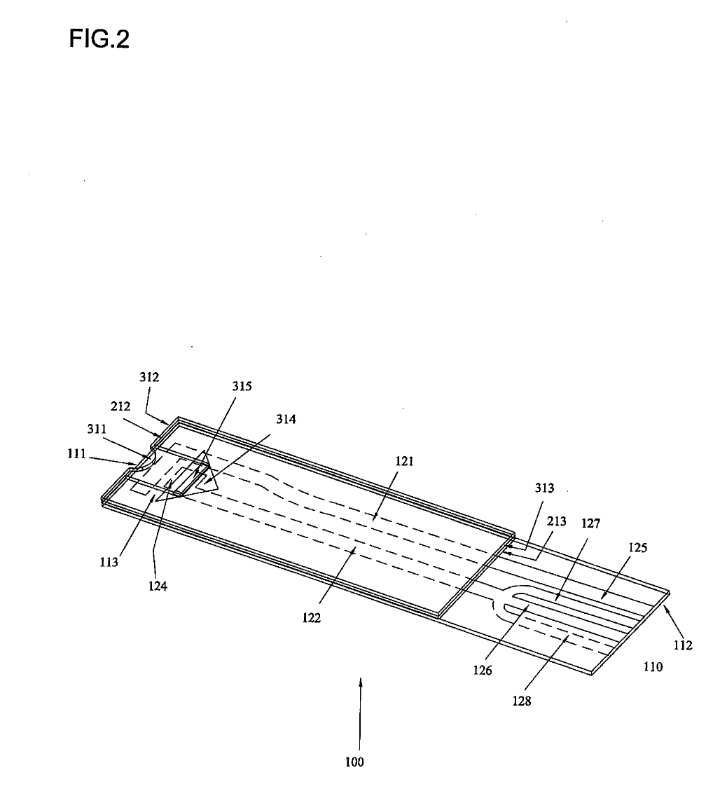

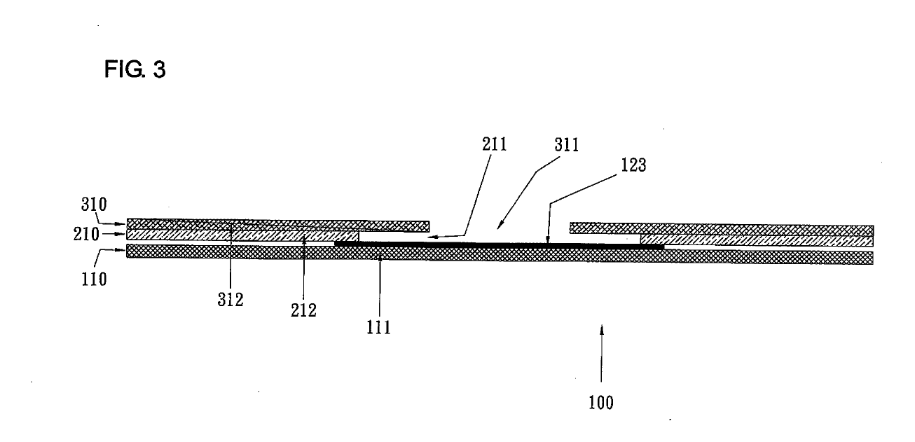

The preferred embodiment of the present invention is shown in FIGS. 1-3. FIG. 1 illustrates an exploded perspective view of an electrochemical biosensor test strip 100 in accordance with the present invention. The electrochemical biosensor test strip 100 is a 3-layer flat elongated construction. Starting from the lowest layer, the electrochemical biosensor test strip 100 comprises a first insulating base plate 110, then a second insulating base plate 210, and finally a cover 310. These layers 110, 210, and 310 can be made from the materials with electrically insulating property. Examples of a preferred material are polyvinyl chloride, polycarbonate, polyethylene terephthalate, polyester, polystyrene, polybutadiene, polyurethane and polyimide.

The first insulating base plate 110 preferably takes a rectangular form, which may be defined longitudinally as having two ends extending from a liquid sampling end 111 to an electrical contact end 112. A pair of conductive tracks, 121 and 122, is screen printed on the first insulating base plate 110. Near the liquid sampling end 111 are the working electrode 123 and reference electrode 124. Near the electrical contact end 112 are a plurality of electrical contacts 125, 126, 127 and/or 128. The conductive tracks 121, 122 are separated apart from each other. Of the electrical contacts, at least one electrical contact (127 and/or 128) electrically connected to the electrical contact 126 is a turn-on/type-recognition electrical contact. The turn-on electrical contact is used to wake up a meter (an apparatus to read the strip information; not shown) automatically when the test strip is inserted into the meter. This is used for the purpose of saving power when the battery powered meter is not in use. The type-recognition electrical contact is used to recognize the type of the strip (e.g., glucose strip, uric acid strip or cholesterol strip). In other words, it is used to determine if the type of electrochemical biosensor test strip used is corresponding to the analyte to be measured, which can prevent the misusing of strips when a multi-functional meter is in user's hand. Suitable for use in these conductive tracks are materials with electrically conductive property. Examples of a preferred material are copper, silver, gold, platinum, titanium, palladium, and carbon.

The second insulating base plate 210 which overlaid on top of the first insulating base plate 110 is preferably rectangular in shape. A U-shaped cutout 211 is formed in the front end 212 of the second insulating base plate 210. The second insulating base plate 210 has the same width as the first insulating base plate 110. The length of the second insulating base plate 210 is shorter than that of the first insulating base plate 110, which in turn causes an exposure of the electrical contacts 125, 126, 127 and/or 128 when the front end 212 is in alignment with the liquid sampling end 111. The exposure of the electrical contacts 125, 126, 127 and/or 128 is used to contact the corresponding electrical contacts in the meter. The U-shaped cutout 211 of the second insulating base plate 210 not only defines the boundaries of the reaction area 113, but it also exposes a portion of the working electrode 123 and reference electrode 124 of the test strip 100. The preferred width and length of the U-shaped cutout 211 range from 1 mm to 4 mm and from 2 mm to 6 mm, respectively. Overlaying the reaction area 113, the exposed portion of the working electrode 123, and reference electrode 124, is a reagent film used to react with an analyte in a liquid sample.

The reagent film is formed by drying a mixture of reagents containing at least one biological active substance (e.g., enzyme) and other components such as mediator, surfactant, and at least one binder. Enzymes suitable for use are ones that are responsible for the chemical reaction of the analyte. Examples of a preferred enzyme are glucose oxidase or glucose dehydrogenase for measuring glucose. For measuring uric acid, the enzyme is preferably uricase. For measuring cholesterol, the enzymes are preferably cholesterol oxidase and cholesterol esterase. Suitable mediator for use is the material which is capable of undergoing reversible, oxidation-reduction reaction and electron transferring. Examples are potassium ferricyanide, tetrathioflilvalene, phenazine ethosulfate, and hexacyanoferrate, methylene blue, benzoquinone, and phenyidiamines, 3,3′,5,5′-tetramethylbenzidine. Surfactant suitable for use is preferably selected, either individually or in combination, from the group including Triton X-100, cholic acid, polyethylene glycol, t-octylphenoxypolyethoxyethanol, sodium lauryl sulfate, polyoxyethylenesorbitan monolaurate (Tween 20, Tween 40, Tween 60, Tween 80). Binders suitable for use are preferably selected, either individually or in combination, from the group including polyvinyl alcohol, polyvinyl pyrrolidone, polyethylene glycol, gelatin and methyl cellulose.

The cover 310 is preferably rectangular in shape. The preferred width and length for the cover 310 is the same as the second insulating base plate 210. When the cover 310 is placed on top of the second insulating base plate 210, a reaction chamber is formed. The size of the reaction chamber is defined by the reaction area 113 and the thickness of the second insulating base plate 210, which in turn defines the volume of liquid sample required per test strip. The reaction chamber of the present invention requires a lower volume of liquid sample which is about 2.5 microliters. The action of capillary force for introducing the liquid sample into the reaction chamber is also defined by the thickness of the second insulating base plate. Acceptable thicknesses of the second insulating base plate 210 is in the range of 0.1 mm to 0.5 mm. A semicircle-shaped cutout 311 formed in the front end 312 of the cover 310 is used to accommodate a drop of user's body fluid (e.g., finger blood) when the front end 312 is in alignment with the front end 212 of the second insulating base plate 210 and the liquid sampling end 111 of the first insulating base plate 110. In other words, the liquid sample is introduced into the reaction chamber from the small semicircle-shaped cutout 311 on the front end 312 of the cover 310. The cover 310 has an open slot 315 within a picture of reverse triangle 314. The picture of reverse triangle 314 is used as a direction instruction for inserting the test strip 100. The open slot 315 may be used as an air vent opening to reduce the air bubbles trapped in the reaction chamber during the introduction of liquid sample. On the other hand, the open slot 315 may also be used as an indicator to insure that the reaction chamber is filled with liquid sample if the open slot 315 displays the color of the liquid sample.

### EXAMPLES

According to the present invention, a method for fabricating such a electrochemical biosensor test strip is provided comprising the steps of:

Step 1: Screen printing conductive tracks (working electrode 123 and reference electrode 124 with a plurality of electrical contacts 125, 126, 127 and/or 128) on the first insulating base plate 110. The conductive materials for printing such as copper, silver, gold, platinum, titanium, palladium, and carbon may be selected either individually or in combination.

Step 2: After the printed conductive material is dried, a second insulating base plate 210 with a U-shaped cutout 211 in the front end 212 is placed on top of the first insulating base plate 110. The U-shaped cutout 211 of the second insulating base plate 210 defines the boundaries of the reaction area 113, and exposes a portion of the working electrode 123 and reference electrodes 124 of the test strip 100.

Step 3: Overlaying the reaction area 113 is a reagent film which contains at least one biological active substance (e.g., enzyme) and other components such as mediator, surfactant, and at least one binder. Examples of the formula of the reagent film are described as follows:

Formula 1 (for determination of glucose):

| | |
|---|---|
| Phosphate buffer (pH = 6.5) | 87.0% |
| Potassium ferricyanide | 10.0% |
| Glucose oxidase | 1.2% |
| Methyl cellulose | 1.0% |
| Triton X-100 | 0.6% |
| Bovine serum albumin | 0.2% |

Formula 1-1 (for determination of glucose):

| | |
|---|---|
| Phosphate buffer (pH = 6.5) | 87.0% |
| Potassium ferricyanide | 10.3% |
| Glucose oxidase | 1.4% |
| PEG | 0.6% |
| Tween 20 | 0.4% |
| Bovine serum albumin | 0.3% |

Formula 1-2 (for determination of glucose):

| | |
|---|---|
| Phosphate buffer (pH = 6.5) | 87.0% |
| Potassium ferricyanide | 10.0% |
| Glucose oxidase | 1.2% |
| Carboxymethyl cellulose | 1.0% |
| Triton X-100 | 0.6% |
| Bovine serum albumin | 0.2% |

Formula 2 (for determination of uric acid):

| | |
|---|---|
| Phosphate buffer (pH = 7.0) | 92.4% |
| Potassium ferricyanide | 6.0% |
| Uricase | 0.4% |
| Methyl cellulose | 0.6% |
| Triton X-100 | 0.5% |
| Bovine serum albumin | 0.1% |

Formula 3 (for determination of cholesterol):

| | |
|---|---|
| Phosphate buffer (pH = 7.0) | 84.8% |
| Potassium ferricyanide | 10.0% |
| Cholesterol esterase | 1.0% |
| Cholesterol oxidase | 2.0% |
| Methyl cellulose | 1.0% |
| Triton X-100 | 1.0% |
| Bovine serum albumin | 0.2% |

Step 4: Placing a cover 310 on top of the second insulating base plate 210 and aligning two front ends 312, 212—an electrochemical biosensor test strip is ready for use.

When a liquid sample is introduced to a single electrochemical biosensor test strip of the present invention, the liquid sample enters the reaction chamber from the small semicircle-shaped cutout 311 on the front end 312 of the cover 310 and stops at bottom end of the U-shaped cutout 211 of the second insulating base plate 210. When the color of the liquid sample is shown on the open slot 315, the reaction chamber is filled with liquid sample. The volume of the liquid sample required for the electrochemical biosensor test strip is about 2.5 microliters.

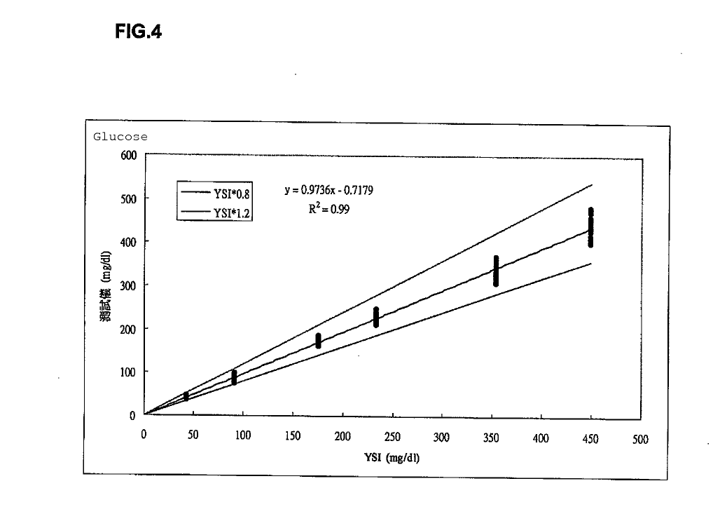

The following example illustrates the correlation of glucose concentration readings determined by the test strips of the present invention and by a traditional YSI glucose analyzer. To make clinically relevant blood glucose concentrations, a venous blood sample was collected, separated into 6 tubes, and each tube was spiked with different glucose concentrations (43, 91, 176, 234, 354, and 449 mg/dL). These blood glucose concentrations were determined by a YSI glucose analyzer. Using the eBsensor glucose meter (not shown), the coefficients of variation (CVs) calculated from the 30 measurements of each glucose concentration were found to be 5.87%, 5.46%, 3.99%, 4.90%, 4.64%, and 5.61%, respectively. The glucose concentration readings using electrochemical biosensor test strips of the present invention plotted against the concentration values determined by the YSI analyzer are illustrated in FIG. 4. A regression coefficient of 0.99 indicated a linear relationship of glucose concentration readings using the test strips of the present invention versus glucose concentration readings determined by the YSI analyzer.

### REFERENCES

All references are listed herein for the convenience of the reader. Each is incorporated by reference in its entirety.
1. U.S. Pat. No. 5,120,420 Biosensor and a process for preparation thereof, (Nankai et al. 1992).
2. U.S. Pat. No. 5,288,636 Enzyme electrode system, (Pollmann et al., 1994)
3. U.S. Pat. No. 5,437,999 Electrochemical sensor, (Diebold et al. 1995)
4. U.S. Pat. No. 5,727,548 Strip electrode with screen printing, (Hill et al. 1998)
5. U.S. Pat. No. 5,762,770 Electrochemical biosensor test strip, (Pritchard et al. 1998)
6. Alto et al. (2002) J Am Board Fam Pract 15:1-6,
7. Arens et al. (1998) Clin Chem Lab Med 36: 47-52;
8. Dai et al. (2004) Clin Chim Acta. 349:135-41).
9. Demers et al. (2003) Am J Health Syst Pharm 60:1130-1135;
10. Evans (1999) BMJ 319: 83-86;
11. Franciosi (2001) Diabetes Care 24: 1870-1877
12. Ikeda and Tsuruoka (1994) Diabetes Res Clin Pract Suppl: S269-271;
13. Kilpatrick et al. (1994) Diabet Med 11: 214-217;
14. Rheney and Kirk (2000) Ann Pharmacother 34: 317-321;
15. Solnica et al. (2001) Clin Chem Lab Med 39:1283-1286;
16. Sonksen et al. (1978) Lancet 1: 729-732;
17. Tate et al. (1992) Diabetes Care 15: 536-538;
18. Trajanoski et al. (1996) Diabetes Care 19: 1412-1415;
19. Walford et al. (1978) Lancet 1: 732-735;

What is claimed is:

1. An electrochemical biosensor test strip for the determination of an analyte in a liquid sample, which comprises:
a first insulating base plate having a liquid sampling end and an electrical contact end;
a pair of conductive tracks formed on said first insulating base plate;
a reaction area formed near said liquid sampling end of said first insulating base wherein a reagent film is deposited in said reaction area;
a second insulating base plate wherein a cutout of said second insulating base plate overlays said reaction area; and
a cover disposed atop said second insulating base plate.

2. The electrochemical biosensor test strip of claim 1, wherein said base plate material is selected individually or in combination from the group consisting of polyvinyl chloride, polycarbonate, polyethylene terephthalate, polyester, polystyrene, polybutadiene, polyurethane and polyimide.

3. The electrochemical biosensor test strip of claim 1, wherein said conductive track material is selected individually or in combination from the group consisting of copper, silver, gold, platinum, titanium, palladium, and carbon.

4. The electrochemical biosensor test strip of claim 1, wherein said conductive tracks are separated apart from each other.

5. The electrochemical biosensor test strip of claim 1, wherein said pair of conductive tracks are a working electrode and a reference electrode located near said liquid sampling end and a plurality of electrical contacts are located near said electrical contact end.

6. The electrochemical biosensor test strip of claim 5, wherein said plurality of electrical contacts are electrically connected together.

7. The electrochemical biosensor test strip of claim 6, wherein at least one of said plurality of electrical contacts is a wake-up/type-recognition electrical contact used to turn on a meter and to recognize the type of the strip.

8. The electrochemical biosensor test strip of claim 1, wherein said cutout defines the boundaries of said reaction area and exposes a portion of said conductive tracks.

9. The electrochemical biosensor test strip of claim 1, wherein said reagent film comprises at least one biological active substance and other components including mediator, surfactant, and at least one binder.

10. The electrochemical biosensor test strip of claim 9, wherein said biological active substance is glucose oxidase for measuring glucose.

11. The electrochemical biosensor test strip of claim 9, wherein said biological active substance is uricase for measuring uric acid.

12. The electrochemical biosensor test strip of claim 9, wherein said biological active substances are cholesterol oxidase and cholesterol esterase for measuring cholesterol.

13. The electrochemical biosensor test strip of claim 9, wherein said mediator is selected individually or in combination from a group consisting of potassium ferricyanide, tetrathioflilvalene, phenazine ethosulfate, and hexacyanoferrate, methylene blue, benzoquinone, phenyidiamines, and 3,3′,5,5′-tetramethylbenzidine.

14. The electrochemical biosensor test strip of claim 9, wherein said surfactant is selected individually or in combination from a group consisting of Triton X-100, cholic acid, polyethylene glycol, t-octylphenoxypolyethoxyethanol, sodium lauryl sulfate, and polyoxyethylenesorbitan monolaurate.

15. The electrochemical biosensor test strip of claim 9, wherein said binder is selected individually or in combination from a group consisting of polyvinyl alcohol, polyvinyl pyrrolidone, polyethylene glycol, gelatin and methyl cellulose.

16. The electrochemical biosensor test strip of claim 1, wherein the length of said second insulating base plate and the length of said cover are shorter than that of said first insulating base plate in order to expose said electrical contacts.

17. The electrochemical biosensor test strip of claim 1, wherein said cover has a cutout and an open slot.

18. A method for fabricating an electrochemical biosensor test strip comprises the steps of:
(a) forming separated conductive tracks on a first insulating base plate that has a liquid sampling end and an electrical contact end;
(b) overlaying a second insulating base plate on top of said first insulating base plate;
(c) defining a reaction area on said liquid sampling end of said first insulating base plate with a cutout of said second insulating base plate;
(d) coating a mixture of reagent to form a reagent film on top of said reaction area, wherein said mixture comprises at least one biological active substance and other components including mediator, surfactant, and at least one binder;
(e) selecting at least one biological active substance from a group consisting of oxidase, uricase, cholesterol oxidase, and cholesterol esterase;
(f) overlaying a cover on top of said second insulating base plate, wherein the length of said second insulating base plate and the length of said cover are shorter than that of said first insulating base plate;

19. The method of claim 18, wherein said mediator is selected individually or in combination from a group consisting of potassium ferricyanide, tetrathioflilvalene, phenazine ethosulfate, and hexacyanoferrate, methylene blue, benzoquinone, and phenyidiamines, 3,3′,5,5′-tetramethylbenzidine; said surfactant is selected individually or in combination from a group consisting of Triton X-100, cholic acid, polyethylene glycol, t-octylphenoxypolyethoxyethanol, sodium lauryl sulfate, polyoxyethylenesorbitan monolaurate; and said binder is selected individually or in combination from a group consisting of polyvinyl alcohol, polyvinyl pyrrolidone, polyethylene glycol, gelatin and methyl cellulose.
